(19) United States
(12) Patent Application Publication
(10) Pub. No.: US 2003/0097033 A1
(43) Pub. Date: May 22, 2003

(54) Method for the sidechain alkylation of alkylbenzenes

(76) Inventor: Ulrich Steinbrenner, Neustadt (DE)

Correspondence Address: KEIL & WEINKAUF, 1350 CONNECTICUT AVENUE, N.W., WASHINGTON, DC, 20036, US

(21) Appl. No.: 10/258,944

(22) PCT Filed: May 8, 2001
(86) PCT No.: PCT/EP01/05217

(30) Foreign Application Priority Data
May 9, 2000 (DE) 10022439/3
May 15, 2000 (DE) 10023771.1

(51) Int. Cl. C07C 2/66

(52) U.S. Cl. 585/462; 502/344; 585/446

(57) ABSTRACT

The present invention relates to a process for the preparation of an alkali metal catalyst by mixing a melt of the alkali metal with a pulverulent, solid inorganic material at above the melting point of the alkali metal, wherein the pulverulent, solid inorganic material comprises a mixture of potassium carbonate and at least one alkali metal chloride selected from sodium chloride and potassium chloride.

The present invention also relates to a process for the preparation of the catalysts employed in the above-mentioned process.

### DESCRIPTION

The present invention relates to a process for the side-chain alkylation of alkylbenzenes I which have at least one alkyl side chain containing an α-hydrogen atom, by reaction of an alkylbenzene I with a monoolefin in the presence of an alkali metal catalyst.

It is known that alkylaromatic compounds having an active hydrogen atom on the α-carbon atom of the alkyl chain (benzylic hydrogen atom) couple to the α-carbon atom of olefins in the presence of alkali metals. This process is also known as side-chain alkylation. The alkali metals employed are frequently sodium, potassium or sodium/potassium alloy. Owing to the comparatively low selectivity of the alkali metal for this reaction, however, by-products are frequently formed. Besides the formation of isomeric alkylaromatic compounds, which can frequently only be separated off from the desired target compound with difficulty, the cyclization of the alkylaromatic compounds formed primarily and dimerization of the olefins employed is also observed. Thus, for example, in the reaction of toluene with propene in the presence of alkali metals, n-butylbenzene, methylindanes and diverse hexene isomers are found in addition to the desired isobutylbenzene. The low catalytic activity of the alkali metal catalysts, with the consequence of low space-time yields, is also problematic.

It has been described in various places in the prior art that the side-chain alkylation is carried out in the presence of alkali metal catalysts in which the alkali metal is in finely divided form on an inorganic support. Potassium carbonate, in particular, has established itself as support here (see, for example, GB 933,253, GB 2,249,737, GB 2,254,802, FR 2,609,024, EP-A 173 335, WO 88/04955, J 61053-229-A, J 61221-133-A and J 61227536-A).

However, the use of alkali metals on potassium carbonate supports only solves the above-mentioned problems to an inadequate extent.

In particular, the space-time yields achieved with these catalysts are frequently inadequate. The selectivity is also not always satisfactory. In addition, the problem exists with these catalysts that tar-like coatings, presumably attributable to the formation of alkali metal salts of acidic hydrocarbons, for example indenes, cyclopentadienes, dihydroanthracenes or 1-alkynes, or to polymerization processes deposited on the walls of the reactor.

WO 91/16284 describes alkali metal catalysts for the reaction of alkylbenzenes with 1,3-butadiene. These alkali metal catalysts are obtained by dispersion of the alkali metal in a suspension of the potassium salt in the alkylaromatic compound. Potassium salts proposed are potassium carbonate, potassium chloride, mixtures thereof and mixtures of potassium carbonate with sodium carbonate and sodium chloride.

It is an object of the present invention to provide a process for the side-chain alkylation of alkylaromatic compounds using monoolefins which is distinguished by good space-time yields and high selectivity.

We have found, surprisingly, that this object can be achieved if the side-chain alkylation is carried out using an alkali metal catalyst in the form of an alkali metal which is finely distributed on an inorganic support material if the inorganic material is a mixture of potassium carbonate and at least one alkali metal chloride selected from sodium chloride and potassium chloride.

The present invention thus relates to a process for the preparation of an alkali metal catalyst by mixing a melt of the alkali metal with a pulverulent, solid inorganic material at above the melting point of the alkali metal, wherein the pulverulent, solid inorganic material comprises a mixture of potassium carbonate and at least one alkali metal chloride selected from sodium chloride and potassium chloride.

The terms “inorganic substance” and “inorganic support material” here and below apply to the inorganic substance employed for the preparation of the catalyst. In the preparation of the catalyst, chemical reactions of the support with the alkali metal can occur, resulting in a chemical change in the support. The present invention naturally also relates to these cases.

Preference is given in accordance with the invention to catalysts in which the alkali metal chloride in the inorganic substance is potassium chloride. In principle, small amounts of other salts, preferably alkali metal salts, can be tolerated in the inorganic substance, where their content generally does not exceed 5% by weight, in particular 1% by weight. In particular, the inorganic substance comprises at least 95% by weight of a mixture of potassium chloride and potassium carbonate. The inorganic substance particularly preferably consists exclusively of potassium carbonate and potassium chloride, apart from the impurities typically present in these salts. It has furthermore proven favorable for the molar ratio between potassium carbonate and alkali metal chloride, in particular potassium chloride, to be in the range from 3:97 to 45:55, corresponding to a K₂CO₃:KCl weight ratio of from 5:95 to 60:40.

It has proven particularly favorable for the alkali metal in the process according to the invention to be sodium, which may comprise up to 5% by weight of other metals, as usually found in technical-grade sodium, for example potassium, calcium or strontium. In particular, use is made of technical-grade sodium, which usually comprises less than 1% by weight of the abovementioned metals as impurities.

The weight ratio between alkali metal and inorganic support material in the alkali metal catalysts used in accordance with the invention is preferably in the range from 1:1 to 1:50, in particular in the range from 1:2 to 1:30 and particular preferably in the range from 1:5 to 1:20.

The catalysts according to the invention can be prepared in the manners known for the preparation of supported alkali metal catalysts. Mention may be made here of the following:

mixing the molten alkali metal with the inorganic substance,

impregnation of the inorganic substance with solutions of an alkali metal azide, drying the mixture and decomposing the alkali metal azide,

vapor deposition of the alkali metal onto the inorganic substance, or

impregnation of the inorganic substance with a solution of the alkali metal in ammonia and removal of the ammonia.

In general, the inorganic substance used for the preparation of the catalyst will comprise only small amounts of water, preferably not more than 2000 ppm and in particular not more than 500 ppm. To this end, the inorganic substance, which is generally prepared by mixing the individual components by methods conventional for this purpose, is subjected to a drying process before treatment with the alkali metal. The inorganic substance is in general warmed for drying to temperatures of ≧100° C., preferably 200° C., in particular above 250° C. and particularly preferably to a temperature in the region of 250° C. to 400° C. In order to support the drying, a reduced pressure can be applied and/or a stream of inert gas can be passed through the inorganic substance.

It has furthermore proven favorable for the inorganic substance used for the preparation of the alkali metal catalyst to have a mean particle size of less than 1000 μm, in particular less than 200 μm and particularly preferably in the range from 10 to 100 μm. In general, use is therefore made of a support material obtained by grinding the components potassium carbonate and alkali metal chloride. The grinding can be carried out in apparatuses conventional for this purpose, such as ball mills, Retsch mills or impact mills.

With respect to the process according to the invention, it has proven particularly favorable to use an alkali metal catalyst obtainable by mixing the molten alkali metal with the solid inorganic substance in powder form at temperatures above the melting point of the alkali metal. Alkali metal catalysts of this type are novel and are likewise a subject matter of the present invention. Use is made, in particular, of a support material which has the composition indicated above as preferred and in particular a support material which has been dried at temperatures of ≧200° C., for example from 250 to 400° C., in a stream of inert gas. The mixing of the alkali metal with the inorganic substance is preferably carried out at a temperature of at least 100° C., preferably at least 150° C. and in particular at least 200° C. A temperature of 500° C. and in particular 400° C. is preferably not exceeded here. To obtain a good support the mixing takes in general at least 30 minutes, preferably at least 60 minutes and particularly at least 90 minutes.

For the mixing of the alkali metal with the inorganic substance, the alkali metal can, for example, be added to the inorganic substance in the form of an extrudate or block and mixed therewith with warming. It is of course also possible to add the pulverulent substance to a melt of the alkali metal. The mixing of the alkali metal with the inorganic substance is carried out in the apparatuses conventional for this purpose, for example in stirred-tank reactors, paddle driers, compounders, edge mills or Discotherm apparatuses.

The mixing of alkali metal and inorganic substance is of course carried out under inert conditions, for example under an inert gas, such as nitrogen or argon, or under an inert-gas mixture, the inert gas generally containing less than 500 ppm of oxygen and less than 100 ppm of water.

If desired, the alkali metal catalyst can, after application of the alkali metal to the inorganic substance, be hydrogenated by treating the mixture of alkali metal and inorganic substance with hydrogen or a mixture of an inert gas and hydrogen at temperatures in the range from 100° C. to 400° C., preferably in the range from 200° C. to 300° C. Then the catalyst is usually cooled and kept under an inert-gas.

In general, the hydrogenation is carried out at atmospheric pressure. The hydrogenation presumably results in the formation of alkali metal hydride catalysts, which likewise catalyze the basic side-chain alkylation. Without being committed to a theory, it is assumed that partial hydrogenation of the catalyst by the hydrogen formed as by-product during the side-chain alkylation occurs in situ under the reaction conditions, even without external supply of hydrogen.

The alkylaromatic compounds I employed are generally derivatives of benzene or of naphthalene which have one, two or three alkyl radicals having 1 to 10 carbon atoms, preferably having 1 to 6 carbon atoms and in particular having 1 to 3 carbon atoms, where at least one of these radicals has a hydrogen atom on an α-carbon atom. Typical alkyl radicals are methyl, ethyl, n-propyl, isopropyl, n-butyl, 2-butyl, isobutyl and n-pentyl. Examples of compounds of this type are mono-, di- and tri-C₁-C₃-alkylbenzenes, such as toluene, xylenes, methylnaphthalenes, mesitylene, ethylbenzenes and isopropylbenzenes, where the two last-mentioned types of compound may also have one or two further methyl groups. Likewise suitable are derivatives of benzene or of naphthalene in which two alkyl radicals, together with the aromatic ring to which they are bonded, form an alicyclic ring, which may, if desired, also contain an oxygen atom. Examples of compounds of this type are 1,2,3,4-tetrahydronaphthalene, indanes and chroman. Preferred alkylaromatic compounds I are derivatives of benzene, in particular those which have one or two alkyl groups. Preferred alkylaromatic compounds have, in particular, at least one methyl group and/or an isopropyl group. Examples of preferred alkylaromatic compounds I are toluene, ortho-xylene, meta-xylene, para-xylene, 1-ethyl-2-methylbenzene, 1-ethyl-3-methylbenzene, 1,2,4-trimethylbenzene, isopropylbenzene and 4-isopropyl-1-methylbenzene.

Of said alkylaromatic compounds I, particular preference is given to toluene, the xylenes and isopropylbenzene. The very particularly preferred alkylaromatic compound I is toluene.

Suitable monoolefins for the process according to the invention are in particular those having 2 to 10 and particularly preferably those having 2 to 5 carbon atoms. Examples thereof are ethene, propene, 1-butene, 2-butene, isobutene, 1-pentene, 2-pentene, 2-methyl-1-butene, 2-methyl-2-butene and 3-methyl-1-butene. Particularly preferred monoolefins are ethene and propene. The process according to the invention can be employed, for example, for the reaction of cumene with ethene to give tert-amylbenzene, toluene with ethene to give n-propylbenzene, for the reaction of xylenes with 1- or 2-butene to give the corresponding tolylpentanes and particular preferably for the reaction of toluene with propene to give isobutylbenzene.

The reaction according to the invention of the monoolefin with the alkylaromatic compound I is generally carried out at elevated temperature, i.e. at temperatures above room temperature, preferably above 80° C. and in particular above 100° C. In general, the reaction temperature in the process according to the invention will not exceed 300° C., preferably 250° C. and in particular 200° C. The reaction is particularly preferably carried out at below 180° C. and very particularly preferably below 160° C., for example at from 120° C. to 140° C.

The process according to the invention can be carried out either in the gas phase or in the liquid phase. The monoolefin can also be introduced in gaseous form into the liquid reaction phase comprising the alkali metal catalyst and the alkylaromatic compound I. The reaction is preferably carried out in a liquid reaction phase. Besides the starting materials, the liquid reaction phase may also comprise a solvent which is inert under the reaction conditions. Examples thereof are aliphatic and alicyclic hydrocarbons, such as octane, hexane, cyclohexane, cyclooctane and decalin. However, the process is preferably carried out without a solvent, i.e. the liquid reaction phase comprises only the liquid starting components and the alkali metal catalyst.

In general, the process is carried out with exclusion of traces of oxygen and water. The starting materials generally contain less than 1000 ppm and very particularly preferably less than 100 ppm of water. The oxygen content of the starting materials is generally less than 500 ppm and particularly preferably less than 50 ppm. To this end, the water is generally removed from the starting materials by known methods, for example by using desiccants, such as active aluminum oxide, silica gel, molecular sieve or activated carbon, by treatment with metallic sodium or potassium or by freezing out.

If the reaction is carried out in the liquid phase, the reaction can be carried out either under an inert-gas atmosphere or alternatively under the inherent vapor pressure of the liquid reaction phase. However, the reaction is particularly preferably carried out in a completely or virtually completely flooded reactor containing virtually no gas phase. This procedure is particularly preferred in the case of continuous performance of the process.

In the process according to the invention, the monoolefin is preferably employed in a sub-stoichiometric molar amount, based on the alkylaromatic compound I. The molar ratio between monoolefin and alkylaromatic compound preferably does not exceed a value of 0.8, in particular 0.6 and particularly preferably 0.5. However, the molar ratio is at least 0.1, in particular at least 0.2 and particularly preferably at least 0.3. Through this measure, dimerization of the monoolefin and secondary reactions of the alkylaromatic compound formed during the reaction, which may also contain active α-hydrogen atoms, are prevented. It is also possible to employ an excess of monoolefin, based on the alkylaromatic compound I, in the process according to the invention, in particular if an alkylaromatic compound which no longer has an α-hydrogen atom is formed in the process according to the invention, for example the tert-amylbenzene formed on reaction of isopropylbenzene with ethene.

The process according to the invention can be designed as a batch process and as a continuous process.

In the batch method, a procedure is generally followed in which the alkylaromatic compound and the alkali metal catalyst are initially introduced, and the monoolefin, preferably in liquid form, is added thereto under reaction conditions at the rate at which it is consumed. In this way, it is achieved that the monoolefin is present in the reaction mixture in a sub-stoichiometric amount, based on the alkylaromatic compound I. When the desired conversion has been reached, the reaction is terminated by cooling the reaction mixture, the alkali metal catalyst is separated off, and the product is worked up in the manner customary for this purpose, preferably by distillation.

The process according to the invention is preferably carried out continuously. To this end, the starting materials are passed continuously under reaction conditions through a reaction zone charged with the catalyst. The alkali metal catalyst can be present in the reaction zone in the form of a fixed bed. Preferably, however, it is in the form of a suspension in the liquid reaction phase. For this purpose, the liquid reaction phase is preferably stirred vigorously, for example using impeller turbines or using anchor stirrers, at speeds of preferably >500 rpm and in particular >800 rpm.

In the continuous embodiment of the process according to the invention, the starting materials can be fed into the reactor either in a single stream or in separate streams. The rate at which the starting materials are fed into the reactor (feed rate) naturally depends upon the reactivity of the starting materials and of the catalyst. The feed rate is preferably in the range from 0.05 to 5 kg of starting materials per kilogram of catalyst material and hour, in particular in the range from 0.1 to 1 kg/h per kilogram of catalyst material. In the case of continuous feed of the starting materials, the molar ratio between monoolefin and alkylaromatic compound I is preferably selected to be less than 1, in particular in a range from 1:10 to 1:2 and especially in the range from 1:4 to 2:3.

In order to recover the target product from the liquid reaction phase, the catalyst is generally separated off from the reaction phase and worked up by distillation. Residues of catalysts remaining in the reaction phase owing to incomplete catalyst removal are generally deactivated before the work-up, for example by addition of water and/or alkanols, such as methanol, ethanol or isopropanol. In the case of a continuous reaction procedure, an amount of liquid reaction phase corresponding to the fed amount is generally discharged from the reactor and worked up in the manner described above. The discharge of the liquid reaction phase is preferably carried out with substantial or complete retention of the alkali metal catalyst in the reaction space. The catalyst is retained, for example, by means of substitute filters or separators, such as cross-flow filters, cartridge filters, membranes or settlers.

In the subsequent work-up by distillation, the liquid reaction phase is separated into the target product, by-products, such as the dimerization product of the monoolefin, any solvent and excess alkylaromatic compound. Any excess alkylaromatic compound I obtained is preferably fed back into the process.

The process according to the invention gives the alkylaromatic desired in each case with high selectivity and good space-time yields. In particular, the process according to the invention proves superior to processes using alkali metal catalysts consisting of alkali metal on potassium carbonate. In addition, the catalysts employed in the process according to the invention are distinguished by a longer service life than conventional catalysts based on alkali metal/potassium carbonate. The interfering formation of tar-like by-products (coating formation in the reactor) and of intensely colored by-products is significantly less than in the case of conventional alkali metal catalysts. The high selectivity for the formation of isobutylbenzene compared with the formation of indanes should be emphasized, in particular in the case of the reaction of toluene with propene.

The following examples serve to illustrate the invention.

I. Preparation of Catalysts

1. General Preparation Procedure

70 g of inorganic substance (K₂CO₃, KCl or a K₂CO₃/KCl mixture) were ground and dried at 300° C. for 15 hours with stirring in a stream of argon in a Duran glass vessel. The substance was cooled, 10.8 g of metallic sodium (technical grade) were added, and the mixture was again heated at 300° C. for 2 hours with stirring in a stream of argon. The mixture was subsequently cooled, and the resultant solid was suspended in 75 g of absolute toluene with stirring under argon, giving a catalyst suspension.

2. The Following Catalysts Were Prepared and Tested:

Catalyst A: 10.8 g of sodium on 70 g of potassium carbonate (not according to the invention).

Catalyst B: 10.8 g of sodium on a mixture of 35 g of potassium chloride and 35 g of potassium carbonate (according to the invention).

Catalyst C: 10.8 g of sodium on 70 g of potassium chloride (not according to the invention).

II. Reaction of Toluene With Propene

1. General Procedure

The reaction was carried out continuously in a stirred-tank reactor having an internal capacity of 270 ml which was fitted with a magnetically coupled stirrer with impeller turbine. The reactor in each case contained the catalyst suspension and was flooded with the mixture of liquid propene and toluene before commencement of the reaction. The reactor was heated to 130° C. and stirred at speeds in the range from 1000 to 1200 rpm. 0.132 mol/h of dry liquid propene and 0.316 mol/h of dry toluene were fed continuously into the reactor. The reaction product was discharged through a 4 μm filter and analyzed for the contents of the products by on-line gas chromatography.

Tables 1 to 3 below show the results for run times in the range from 10 to 100 hours.

### 2. COMPARATIVE EXAMPLE 1

### Reaction With Catalyst A in Accordance With the General Procedure

| Run time | | Selectivity²⁾ [mol %] | | | |
|---|---|---|---|---|---|
| [h] | STY¹⁾ | T → IBB | T → nBB | T → I | P → IBB |
| 10 | 0.016 | 88 | 10.2 | 0.6 | 30 |
| 20 | 0.079 | 88 | 10.6 | 0.6 | 75 |
| 30 | 0.088 | 88 | 10.6 | 0.6 | 77 |
| 40 | 0.091 | 88 | 10.4 | 0.7 | 78 |
| 50 | 0.084 | 88 | 10.0 | 0.8 | 78 |
| 60 | 0.074 | 88 | 9.6 | 0.9 | 76 |
| 70 | 0.070 | 89 | 9.3 | 1.1 | 76 |
| 80 | 0.063 | 89 | 8.8 | 1.3 | 76 |
| 90 | 0.050 | 89 | 8.4 | 1.6 | 76 |
| 100 | 0.046 | 89 | 8.1 | 1.7 | 76 |

T = toluene, IBB = isobutylbenzene, nBB = n-butylbenzene, I = indane, P = propene, cat = catalyst, GC = gas chromatogram

¹⁾ STY=space-time yield in g of (IBB)/(g(cat)·h)

²⁾ Selectivity calculated from GC peak area %, based on the relative peak area corresponding to the proportion in % by weight.

### 2. EXAMPLE 1

### Reaction With Catalyst B in Accordance With the General Procedure

| Run time | | Selectivity²⁾ [mol %] | | | |
|---|---|---|---|---|---|
| [h] | STY¹⁾ | T → IBB | T → nBB | T → I | P → IBB |
| 10 | 0.017 | 88 | 10.9 | 0.4 | 73 |
| 20 | 0.091 | 88 | 10.7 | 0.4 | 78 |
| 30 | 0.098 | 88 | 10.5 | 0.4 | 78 |
| 40 | 0.102 | 88 | 10.3 | 0.5 | 79 |
| 50 | 0.106 | 88 | 10.0 | 0.5 | 79 |
| 60 | 0.103 | 88 | 9.5 | 0.6 | 79 |
| 70 | 0.100 | 88 | 9.2 | 0.8 | 80 |
| 80 | 0.098 | 88 | 8.7 | 1.1 | 80 |
| 90 | 0.096 | 89 | 8.1 | 1.4 | 81 |
| 100 | 0.090 | 89 | 7.4 | 1.6 | 81 |

T = toluene, IBB = isobutylbenzene, nBB = n-butylbenzene, I = indane, P = propene, cat = catalyst, GC = gas chromatogram

¹⁾ STY=space-time yield in g of (IBB)/(g(cat)·h)

²⁾ Selectivity calculated from GC peak area %, based on the relative peak area corresponding to the proportion in % by weight.

### 3. COMPARATIVE EXAMPLE 2

### Reaction With Catalyst C in Accordance With the General Procedure

| Run time | | Selectivity²⁾ [mol %] | | | |
|---|---|---|---|---|---|
| [h] | STY¹⁾ | T → IBB | T → nBB | T → I | P → IBB |
| 10 | 0.004 | 89 | 10.1 | 0.0 | 60 |
| 20 | 0.012 | 88 | 11.3 | 0.4 | 63 |
| 30 | 0.016 | 89 | 10.8 | 0.2 | 65 |
| 40 | 0.017 | 89 | 10.7 | 0.2 | 63 |
| 50 | 0.018 | 89 | 10.6 | 0.2 | 64 |
| 60 | 0.020 | 89 | 10.5 | 0.2 | 63 |
| 70 | 0.021 | 89 | 10.4 | 0.2 | 62 |
| 80 | 0.022 | 89 | 10.3 | 0.2 | 62 |
| 90 | 0.022 | 89 | 10.3 | 0.2 | 62 |
| 100 | 0.023 | 89 | 10.2 | 0.2 | 62 |

T = toluene, IBB = isobutylbenzene, nBB = n-butylbenzene, I = indane, P = propene, cat = catalyst, GC = gas chromatogram

¹⁾ STY=space-time yield in g of (IBB)/(g(cat)·h)

²⁾ Selectivity calculated from GC peak area %, based on the relative peak area corresponding to the proportion in % by weight.

We claim:

1. A process for the side-chain alkylation of alkylbenzenes I which have at least one alkyl side chain containing an α-hydrogen atom, by reaction of the alkylbenzene I with a monoolefin in the presence of an alkali metal catalyst comprising a mixture of an alkali metal and an inorganic substance as support, wherein the inorganic substance is a mixture of potassium carbonate and at least one alkali metal chloride selected from sodium chloride and potassium chloride.

2. A process as claimed in claim 1, wherein the molar ratio between potassium carbonate and alkali metal halide is in the range from 3:97 to 45:55.

3. A process as claimed in claim 1 or 2, wherein the weight ratio between alkali metal and inorganic substance in the catalyst is in the range from 1:1 to 1:50.

4. A process as claimed in one of the preceding claims, wherein the alkali metal is sodium.

5. A process as claimed in one of the preceding claims, wherein an alkali metal catalyst is employed which is obtainable by mixing a melt of the alkali metal with the pulverulent, solid inorganic substance at above the melting point of the alkali metal.

6. A process as claimed in one of the preceding claims, wherein the reaction of the monoolefin with an alkylaromatic compound is carried out at a temperature in the range from 100° C. to 200° C.

7. A process as claimed in one of the preceding claims, wherein the catalyst is in the form of a suspension in the reaction mixture during the reaction.

8. A process as claimed in one of the preceding claims, wherein the monoolefin is employed in a sub-stoichiometric molar amount, based on the alkylaromatic compound I.

9. A process as claimed in one of the preceding claims, wherein the monoolefin is propene and the alkylaromatic compound I is toluene.

10. A process as claimed in claim 1 in a continuous variant, wherein the starting materials are fed into the reactor at a feed rate of from 0.05 to 5 kg per kilogram of catalyst material.

11. A process for the preparation of an alkali metal catalyst by mixing a melt of the alkali metal with a pulverulent, solid inorganic material at above the melting point of the alkali metal, wherein the pulverulent, solid inorganic material comprises a mixture of potassium carbonate and at least one alkali metal chloride selected from sodium chloride and potassium chloride.

12. A process as claimed in claim 11, wherein a pulverulent inorganic substance is employed which is obtainable by drying an intimate mixture comprising potassium carbonate and the alkali metal chloride at temperatures of ≧200° C. in a stream of inert gas.

13. A process as claimed in claim 11, wherein the mixing is carried out at a temperature above 200° C.

14. A process as claimed in claim 11, wherein the catalyst is treated with hydrogen after the mixing.

15. An alkali metal catalyst obtainable by a process as claimed in claims 11 to 14.
